(19) United States
(12) Patent Application Publication
(10) Pub. No.: US 2010/0061935 A1
(43) Pub. Date: Mar. 11, 2010

(54) METHODS OF USING SUSTAINED RELEASE AMINOPYRIDINE COMPOSITIONS

(75) Inventors: Andrew R. Blight, Mahopac, NY (US); Ron Cohen, Irvington, NY (US)

Correspondence Address: Pepper Hamilton LLP, 500 Grant Street, 50th Floor, Pittsburgh, PA, 15219, US

(73) Assignee: ACORDA THERAPEUTICS, INC., Hawthorne, NY (US)

(21) Appl. No.: 12/557,015

(22) Filed: Sep. 10, 2009

Related U.S. Application Data
(60) Provisional application No. 61/095,797, filed on Sep. 10, 2008.

(51) Int. Cl. A61K 31/4409; A61K 38/21; A61K 39/395; A61K 49/00; A61P 25/00

(52) U.S. Cl. 424/9.2; 514/352; 424/85.4; 424/133.1

(57) ABSTRACT

Disclosed herein are methods and compositions related to use of aminopyridines, such as fampridine, to improve impairments of patients with a demyelinating condition, such as MS.

### CROSS REFERENCES

This application claims priority to U.S. Provisional Application No. 61/095,797, filed on Sep. 10, 2008, the entire contents of which is hereby incorporated by reference for any purpose. This application incorporates by reference the following applications: U.S. Provisional Application No. 60/453,734 filed Mar. 17, 2003; 60/528,593, 60/528,592 and 60/528,760 each filed Dec. 11, 2003; U.S. application Ser. No. 11/010,828 filed Dec. 15, 2005; U.S. Provisional Application No. 60/560,894 filed Apr. 9, 2004 and U.S. application Ser. No. 11/102,559 filed Apr. 8, 2005.

### SUMMARY OF THE INVENTION

Not applicable

### BRIEF DESCRIPTION OF THE DRAWINGS

The following drawings form part of the present specification and are included to demonstrate certain aspects of the present disclosure in greater detail. The invention may be better understood by reference to one of these drawings in combination with the detailed description of specific embodiments presented herein.

FIG. 1 shows information regarding fampridine.

FIG. 2 is a flowchart depicting the study design.

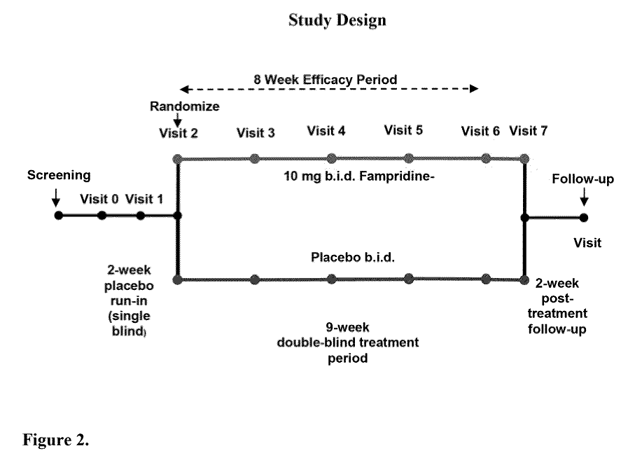

FIG. 3 is a flowchart depicting disposition of patients.

FIG. 4. is a graph depicting the timed walk response rate across treatment groups.

FIG. 5 is a graph depicting timed walk response rates across course types.

FIG. 6. is a graph depicting change in walking speed by timed walk responder analysis group.

FIG. 7 is a graph depicting the change from baseline in lower extremity strength (LEMMT Score).

### DETAILED DESCRIPTION OF THE INVENTION

The present invention relates to methods of using aminopyridine to treat symptoms associated with Multiple Sclerosis (MS). In some embodiments, sustained release-4-aminopyridine is administered to a patient suffering from MS-induced ambulatory deficits. In some embodiments, sustained release-4-aminopyridine is administered to a patient suffering from MS to improve symptoms selected from walking speed, balance, leg strength and combinations thereof. In some embodiments, the invention relates to use of sustained release 4-aminopyridine to improve or stabilize the patients with Multiple Sclerosis (MS).

MS is thought to be an autoimmune disease and is characterized by areas of demyelination (lesions) in the CNS. This characteristic demyelination and associated inflammatory response lead to abnormal impulse conduction or conduction block in nerve fibers traversing the lesions. Lesions can occur throughout the CNS but certain sites such as the optic nerve, brainstem, spinal cord, and periventricular region seem particularly vulnerable. Impaired action potential conduction is probably the major contributor to the symptoms most often reported (e.g., paralysis, visual abnormalities, muscle weakness, nystagmus, sensory abnormalities, and speech disturbances).

Studies of fampridine (4-aminopyridine) have been conducted using intravenous (i.v.) administration and immediate-release (IR) oral capsule formulations in addition to controlled-release or sustained-release formulations. Administration of IR capsules resulted in rapid and short-lasting peaks of fampridine in the plasma. Early pharmacokinetic studies were conducted using an immediate release (IR) formulation for oral administration, which consisted of fampridine powder in a gelatin-based capsule or oral solution. Administration resulted in rapidly changing fampridine plasma levels that were not well tolerated. A sustained-release matrix tablet (Fampridine-SR) was then developed. The Fampridine-SR matrix tablet showed improved stability and an appropriate pharmacokinetic profile for twice-daily dosing.

Studies in people with multiple sclerosis (MS), including Phase 1, 2 and 3 clinical trials, indicate that the drug fampridine improves a variety of neurological functions that are impaired by this disease, with particular attention focused on the effects of the drug to improve ambulation and leg strength.

There remains a need in the art for methods of ameliorating the problem of brain effects such as cognitive impairment in MS, as well as in other patient populations subject to demyelinating and traumatic conditions. There remains a need in the art for methods of treating MS and symptoms of MS. There also remains a need for creating appropriate clinical trials for evaluating the efficacy and/or safety of drugs, including, for example Fampridine-SR, in patients with MS.

Disclosed herein are methods of treating multiple sclerosis in a subject comprising administering a sustained release composition comprising 10 milligrams or less of 4-aminopyridine twice daily to said subject.

Disclosed herein is a method of treating relapsing-remitting multiple sclerosis in a subject comprising administering a sustained release composition comprising 10 milligrams or less of 4-aminopyridine twice daily to said subject.

Disclosed herein is a method of treating secondary progressive multiple sclerosis in a subject comprising administering a sustained release composition comprising 10 milligrams or less of 4-aminopyridine twice daily to said subject.

Disclosed herein is a method of treating primary progressive multiple sclerosis in a subject comprising administering a sustained release composition comprising 10 milligrams or less of 4-aminopyridine twice daily to said subject.

Disclosed herein is a method of treating progressive-relapsing multiple sclerosis in a subject comprising administering a sustained release composition comprising 10 milligrams or less of 4-aminopyridine twice daily to said subject.

In some embodiments, 10 milligrams of 4-aminopyridine is administered twice daily to said subject.

In some embodiments, 5 milligrams of 4-aminopyridine is administered twice daily to said subject.

In some embodiments, the sustained release aminopyridine composition comprises one or both of 3-hydroxy-4-aminopyridine and 3-hydroxy-4-aminopyridine sulfate.

In some embodiments, the sustained release aminopyridine composition is administered every 12 hours during the treatment period.

Also disclosed is a method of treating multiple sclerosis in a subject comprising administering a sustained release composition comprising 10 milligrams or less of 4-aminopyridine twice daily and an immunomodulator to said subject. In some embodiments, the immunomodulator is selected from interferons, natalizumab and glatiramer acetate.

Further disclosed is a method of treating spasticity associated with multiple sclerosis in a subject comprising administering a sustained release composition comprising 10 milligrams or less of 4-aminopyridine twice daily to said subject, wherein the spasticity of said subject is decreased.

Still further disclosed is a method of treating multiple sclerosis multiple sclerosis in a subject comprising measuring a patient's creatinine clearance; and administering a sustained release composition comprising 10 milligrams or less of 4-aminopyridine twice daily to said subject if said subject's creatinine clearance is greater than or equal to 30 ml/min. The measurement of the patient's creatinine clearance may occur prior to initial administration of said 4-aminopyridine or may occur during a treatment period of said patient. In certain embodiments, the treatment period may be one week or more, two weeks or more, four weeks or more, eight weeks or more, or for an indefinite period of time to provide maintenance therapy to said patient. In certain embodiments, administration of the 4-aminopyridine may be stopped or decreased (either in amount or frequency) if said subject's creatinine clearance is less than 30 ml/min. In certain embodiments, administration of the 4-aminopyridine may be increased (either in amount or frequency) if said subject's creatinine clearance is equal to or greater than 30 ml/min, such that a therapeutically effective amount of 4-aminopyridine could not otherwise be maintained in said patient during steady state or during said treatment period.

In another embodiments, the invention provides a method of treating multiple sclerosis multiple sclerosis in a subject comprising measuring said patient's creatinine clearance; and administering a sustained release composition comprising 4-aminopyridine, wherein the amount and the frequency of administration to said patient is dependent upon the measured creatinine clearance.

In some embodiments, the inventions provides a method of increasing walking speed comprising administering to a patient with multiple sclerosis about 10 milligrams of a sustained release aminopyridine composition twice daily. In some such embodiments, the sustained release aminopyridine composition comprises 4-aminopyridine. In other embodiments, the sustained release aminopyridine composition comprises one or both of 3-hydroxy-4-aminopyridine and 3-hydroxy-4-aminopyridine sulfate.

Some embodiments provide a method of improving lower extremity muscle tone comprising administering to a patient with multiple sclerosis about 10 milligrams of a sustained-release aminopyridine composition twice daily. In some such embodiments, the sustained release aminopyridine composition comprises 4-aminopyridine. In other embodiments, the sustained release aminopyridine composition comprises one or both of 3-hydroxy-4-aminopyridine and 3-hydroxy-4-aminopyridine sulfate.

Further disclosed herein is a method of testing the efficacy of a sustained release composition comprising 4-aminopyridine for treating multiple sclerosis comprising:

assessing potential patients for study, based on particular inclusion and exclusion criteria, excluding patients with creatinine clearance rates below about 30 mL/min.;

assigning known portions of patients to placebo and Fampridine-SR groups, unknown to them or an evaluator in a double-blind study for receipt of “drug”; and

assessing one or more of walking speed, leg strength, and spasticity over the course of 8 weeks of treatment.

In some embodiments, the testing protocol further includes obtaining creatinine clearance rates prior to each assessment.

In some embodiments, spasticity is evaluated prior to leg strength evaluation.

A further embodiment, a method assessing the efficacy of a sustained release composition comprising 4-aminopyridine for treating multiple sclerosis is provided. Such method may include assigning known portions of a sample of patients with multiple sclerosis to placebo and Fampridine-SR groups, unknown to them or an evaluator in a double-blind study for receipt of “drug”; and assessing one or more of walking speed, leg strength, and spasticity for said patients over the course of treatment; wherein the size of said sample of patients shall provide about 90% power and a statistical significance level of 0.05 or lower. The method may further comprise assessing adverse events over the course of treatment. The method may further comprise assessing potential patients for study based on particular inclusion and exclusion criteria, including, for example, an exclusion criteria of a creatinine clearance rate below about 30 ml/min. In such methods, the course of treatment may be about eight weeks.

Other embodiments of the invention are disclosed herein or will be apparent to those of skill in the art in light of the disclosure herein.

In the description, figures and tables herein, a number of terms are used. In order to provide a clear and consistent understanding of the specification and claims, the following definitions are provided:

| Abbreviation or Specialist Term | Explanation |
|---|---|
| ADME | Absorption, distribution, metabolism, and excretion |
| Aₑ | Amount of drug excreted |
| APD₃₀, APD₅₀, APD₉₀ | Action potential duration 30%, 50%, 90% |
| AUC | Area under the concentration-time curve |
| AUC₍₀₋₁₎, AUC_{(0-∞)} or AUC_{(0-inf)} | Area under the plasma concentration versus time curve, to the |
| | last quantifiable level, and extrapolated to infinity |
| AUC₍₀₋₁₂₎, AUC₍₀₋₂₄₎ | Area under the plasma concentration versus time curve, 0-12 |
| | hours, 0-24 hours |
| b.i.d. (bid) | Twice daily |
| ¹⁴C | Radioactive carbon 14 |
| CHO | Chinese hamster ovary |
| CI | Confidence interval |
| CL/F | Apparent total body clearance after administration |
| Cl_{R} | Renal clearance |
| cm | Centimeter |
| Cₘₐₓ | Maximum measured plasma concentration |
| CNS | Central nervous system |
| CR | Controlled-release |
| CrCl | Creatinine clearance |
| CumAₑ | Cumulative amount of drug excreted |
| CYP, CYP 450 | Cytochrome p450 isoenzymes |
| ECG | Electrocardiogram |
| EEG | Electroencephalogram |
| F | Female |
| FOB | Functional Observation Battery |
| 4-AP | 4-Aminopyridine |
| g, kg, mg, μg, ng | Gram, kilogram, milligram, microgram, nanogram |
| GABA | Gamma-aminobutyric acid |
| GLP | Good Laboratory Practice |
| h, hr | Hour |
| HDPE | High-density polyethylene |
| hERG | Human ether-a-go-go related gene |
| HPLC | High performance liquid chromatography |
| IC₅₀ | 50% Inhibitory concentration |
| I_{Kr} | Potassium ion channel whose activity is measured in the |
| | hERG assay |
| improvement | Designates an alteration in a parameter in a desired direction. |
| | As used herein, “improvement” also comprises stabilization |
| | of a parameter that would otherwise be deteriorating or |
| | moving in a non-desired direction. |
| IND | Investigational New Drug application |
| IR | Immediate-release |
| i.v. (iv) | Intravenous |
| K⁺ | Potassium |
| Kₑₗ | Elimination constant |
| L, mL | Liter, milliliter |
| LCMS, LC/MS/MS | Liquid chromatography/mass spectrometry |
| LD₅₀ | Median lethal dose |
| Ln | Natural log |
| LOQ | Limit of quantitation |
| M | Male |
| min | Minute |
| mM, μM | Millimolar, micromolar |
| MRT | Mean residence time |
| MS | Multiple sclerosis |
| MTD | Maximum tolerated dose |
| NA | Not applicable |
| ND | None detected |
| NDA | New Drug Application |
| NE | Not evaluable |
| NF | National Formulary |
| NOAEL | No observable adverse effect level |
| NOEL | No observable effect level |
| norm | Normalized |
| NZ | New Zealand |
| pₐₚₚ | Apparent permeability coefficient |
| p.o. | Oral |
| SAE | Serious adverse event |
| SCI | Spinal cord injury |
| SD | Standard deviation |
| sec | Second |
| SEM | Standard error of the mean |
| SPF | Specific pathogen-free |
| SR | Sustained-release |
| SS | Steady state |
| t_{1/2} | Apparent terminal climination half-life |
| t.i.d. (tid) | Three times daily |
| TK | Toxicokinetics |
| TLC | Thin layer chromatography |
| Tₘₐₓ | Time of the maximum measured plasma concentration |
| USP | United States Pharmacopeia |
| UTI | Urinary tract infection |
| V_{d} | Volume of distribution |
| V_{dss} | Volume of distribution at steady state |

When used in conjunction with the word “comprising” or other open language in the claims, the words “a” and “an” denote “one or more.”

Fampridine is a potassium (K+) channel blocker currently being evaluated clinically as a treatment for improving neurological and muscular function in patients with Multiple Sclerosis (MS). Fampridine is the United States Adopted Name (USAN) for the chemical 4-aminopyridine (4 AP), which has a molecular formula of C₅H₆N₂ and molecular weight of 94.1. Both “fampridine” and “4-aminopyridine” will be used throughout this specification to refer to the active drug substance. Fampridine has been formulated as a sustained-release (SR) matrix tablet in various strengths from 5 to 40 mg.

In one embodiment, the following excipients are generally included in each tablet: hydroxypropyl methylcellulose, USP; microcrystalline cellulose, USP; colloidal silicon dioxide, NF; magnesium stearate, USP; and Opadry White. Preferably, the amount of fampridine is 10 milligrams per tablet.

Pharmacologically, the K+ channel blocking properties of 4-aminopyridine and its effects on action potential conduction in demyelinated nerve fiber preparations have been extensively characterized. At low concentrations that are relevant to clinical experience, in the range of 0.2 to 2 μM (18 to 180 ng/mL), 4-aminopyridine is able to block certain voltage-dependent K+ channels in neurons. It is this characteristic that appears to explain the ability of the drug to restore conduction of action potentials in demyelinated nerve fibers. At higher (millimolar) concentrations, fampridine affects other types of K+ channels in both neural and non-neural tissues. Blockade of repolarizing K+ currents can increase synaptic transmission throughout the nervous system by increasing the duration of the pre-synaptic action potential. A range of neurological effects consistent with increased excitability of presynaptic nerve terminals occurs with clinically relevant doses of fampridine.

Effects on Axonal Conduction Block. The K+ channels blocked by low concentrations of 4-aminopyridine are partially responsible for repolarization of neuronal action potentials. These appear to include those found under the myelin sheath in myelinated nerve fibers of adult mammals. These channels are located primarily in the paranodal and internodal membrane of the axon (Waxman and Ritchie, 1993) where they are not significantly activated by the passage of an action potential because the myelin sheath acts as an electrical shield. Therefore, the action potential of normal adult myelinated axons shows little or no sensitivity to 4-aminopyridine at concentrations below 100 μM (9.4 μg/mL) (Shi and Blight, 1997). Concentrations above 1 mM (94.1 μg/mL) tend to cause gradual depolarization of the axon resting potential, perhaps by interacting with leakage channels (Shi and Blight, 1997).

When the axon is demyelinated, the internodal membrane and its ion channels become exposed to larger electrical transients during the action potential. Leakage of ionic current through the K+ channel, under these conditions, can contribute to the phenomenon of action potential conduction block (Waxman and Ritchie, 1993). 4-Aminopyridine may prolong nerve action potentials by blocking these exposed channels and inhibiting repolarization (Sherratt et al., 1980). This is consistent with the ability of the drug to overcome conduction block and increase the safety factor for conduction in some critically demyelinated axons (Bostock et al., 1981; Targ and Kocsis, 1985) including those in chronically injured and partially remyelinated mammalian spinal cord (Blight, 1989; Shi and Blight, 1997). An additional study (Shi et al., 1997) showed that this effect of 4-aminopyridine in the chronically injured spinal cord of guinea pigs occurs at a concentration threshold between 0.2 to 1 μM (19.1 to 94.1 ng/mL), though in this tissue it is most effective at about 10 μM (941 ng/mL).

Repetitive impulse activity, either spontaneous or in response to single stimuli, occurs in some demyelinated axons exposed to higher levels [0.1 to 1 mM (9.4 to 94.1 mg/mL)] of 4-aminopyridine in vitro (Blight, 1989; Bowe et al., 1987; Targ and Kocsis, 1985). A similar effect at lower concentrations on susceptible neurons or nerve endings may explain the paresthesias and pain in the area of intravenous infusion that have been reported as side effects of clinical exposure to 4-aminopyridine in human subjects. However, there are no published data to indicate that repetitive spontaneous activity occurs in such nerve fibers with lower, clinically relevant concentrations in the range of 0.25 to 1 μM (23.5 to 94.1 ng/mL).

It is understood that blockade of K currents amplifies synaptic transmission throughout the brain and spinal cord. A range of neurological effects occurs with increasing concentrations of 4-aminopyridine in the central nervous system (CNS), up to and including the initiation of seizures. Various in vitro brain slice experiments have shown epileptiform discharges in the amygdala (Gean, 1990) and hippocampus (Rutecki et al., 1987) of rats when the tissue was superfused with solutions containing 5 to 500 μM (0.47 to 47 μg/mL) 4-aminopyridine. Seizure activity in animals has been seen following large doses of 4-aminopyridine, and seizure activity is part of the toxicological profile of the drug. Synchronous bursting activity in the spinal of decerebrate cats has been recorded following administration of very large doses of 4-aminopyridine (5 to 20 mg/kg), which would be expected to produce plasma levels in the region of several hundred ng/mL (Dubuc et al., 1986). For the first time herein, these neurological effects are disclosed to be an aspect in the treatment of neuro-cognitive impairment (and related neuro-psychiatric issues), and are overcome by methods in accordance with the invention.

Absorption. 4-Aminopyridine is rapidly absorbed following oral administration. In an in situ study, 4-aminopyridine was more rapidly absorbed from the small intestine than from the stomach. The absorption half-life was 108.8 minutes and 40.2 minutes for the stomach and small intestine, respectively. In an in vitro study with vascularly perfused rat gut segments, the regional apparent permeability coefficient (pₐₚₚ×10⁻⁶ cm/sec) of 4-aminopyridine was high in the upper small intestine (22.7 cm/sec) and decreased distally towards the large intestine (2.9 cm/sec) compared to a poorly permeable marker (atenolol; 1.9 cm/sec in the upper small intestine and 0 cm/sec in the large intestine) (Raoof et al., 1997).

Following oral administration of (non-sustained release) 4-aminopyridine in animals, peak plasma concentrations occur within 1 hour of dosing. Based on comparisons of the areas under the plasma concentration-versus-time curve (AUC_{(0-∞)}) following i.v. and p.o. administration of 4-aminopyridine (2 mg/kg), the bioavailability of 4-aminopyridine was reported to be approximately 66.5% in male rats and 55% in female rats (M 2001-03). Following oral administration, peak plasma concentrations were 38% lower in females than in males, although both (AUC_{(0-∞)}) and body weight were similar; AUC values did not differ between males and females following i.v. administration.

Studies were performed in rats and dogs using ¹⁴C-labeled 4-aminopyridine (1 mg/kg) given as a single oral gavage dose in solution. In both species, 14C 4-aminopyridine was rapidly absorbed. Peak plasma levels were achieved within 0.5 to 1 hour in both species. The peak plasma levels (Cₘₐₓ) and the extent of absorption as reflected by the AUC were both approximately four-fold higher in the dog than in the rat following doses equal on a mg/kg basis. In these studies, there were no gender differences evident in either species. These results are summarized in Table 1.

TABLE 1
Summary of Absorption Data for Rats and Dogs Following Single Oral
Administration of ¹⁴C-4-Aminopyridine 1 mg/kg (Study Nos. HWI 6379-101
and HWI 6379-102)

| | Rats | | Dogs | |
|---|---|---|---|---|
| | (Study HWI 6379-101) | | (Study HWI 6379-102) | |
| Parameter | Males (N = 3¹) | Females (N = 3¹) | Males (N = 3) | Females (N = 3) |
| Cₘₐₓ (μg/g) | 0.189 ± 0.0202 | 0.168 ± 0.0157 | 0.574 ± 0.1230 | 0.635 ± 0.1028 |
| Tₘₐₓ (hr) | 1.0 | 0.5 | 1.0 ± 0 | 0.8 ± 0.3 |
| AUC (μg · hr/mL) | 0.498 ± 0.0176 | 0.506 ± 0.0633 | 2.03 ± 0.406 | 1.92 ± 0.150 |
| t_{1/2} (hr) | 1.1 ± 0.04 | 1.4 ± 0.17 | 2.1 ± 0.14 | 1.8 ± 0.04 |

¹Per time point

When administered orally, fampridine is completely absorbed from the gastrointestinal tract. The absolute bioavailability of two formulations of IR tablets was reported to be 95% (Uges et al., 1982). Absolute bioavailability of Fampridine-SR tablets has not been assessed, but relative bioavailability (as compared to an aqueous oral solution) is 95% Absorption is rapid unless administered in a modified matrix. When a single Fampridine-SR tablet 10 mg dose is administered to healthy volunteers while in a fasted state, mean peak concentrations ranging in different studies from 17.3 ng/mL to 21.6 ng/mL occurred 3 to 4 hours post-administration (Tₘₐₓ). In comparison, the Cₘₐₓ achieved with the same 10 mg dose of a fampridine oral solution was 42.7 ng/mL which occurred approximately 1.1 hours after dose administration. Exposure increases proportionally with dose, and steady state maximum concentrations are approximately 29-37% higher than for single doses.

Table 2 illustrates the dose proportionality of 10 mg and 25 mg single doses and the relative bioequivalence of a solid oral dosage form and oral solution.

TABLE 2
Relative Bioavailability/Bioequivalence Summary Study Results Conducted
in Healthy Adult Volunteers (N = 26 with Data)

| | Dose | | | | | 10 mg vs. 25 mg | |
|---|---|---|---|---|---|---|---|
| | | | Buffered | 10 mg vs. solution | | (dose-adjusted) | |
| | Fampridine SR | | Solution | Ratio of | | Ratio of | |
| | Tablet Dose | | (0.83 mg/mL) | Geometric | | Geometric | |
| Parameter | 10 mg | 25 mg | 10 mg | Means* | 90% CI | Means* | 90% CI |
| In-Cₘₐₓ | 2.91 | 3.77 | 3.73 | 43.6 | 41.07-46.35 | 104.3 | 98.07-110.88 |
| In-AUC₍₀₋ₜ₎ | 5.21 | 6.09 | 5.35 | 86.7 | 80.60-93.26 | 102.1 | 94.96-109.99 |
| In-AUC_{(0-inf)} | 5.37 | 6.17 | 5.42 | 94.7 | 88.23-101.55 | 110.9 | 103.20-119.25 |

The dose proportionality of exposure following single doses of Fampridine-SR is illustrated in Table 3. The pharmacokinetic disposition following of multiple doses of Fampridine-SR is illustrated in Table 4.

TABLE 3
Dose-Normalized Pharmacokinetic Parameter Values (Mean ± SEM)
Following Single Oral Administration of Fampridine-SR Tablets to
Patients with MS

| | Dose (mg) | | | |
|---|---|---|---|---|
| | 5 | 10 | 15 | 20 |
| Parameter | (n = 24) | (n = 24) | (n = 24) | (n = 23) |
| Cₘₐₓ-norm* | 13.1 ± 0.6 | 12.6 ± 0.7 | 12.3 ± 0.7 | 12.3 ± 0.8 |
| (ng/mL) | | | | |
| Tₘₐₓ (hours) | 3.9 ± 0.2 | 3.9 ± 0.3 | 3.6 ± 0.3 | 3.6 ± 0.3 |
| AUC-norm* | 122.1 ± 9.4 | 122.1 ± 9.4 | 131.5 ± 7.4 | 127.8 ± 6.9 |
| (ng · hr/mL) | | | | |
| t_{1/2} (hours) | 5.8 ± 0.5 | 5.6 ± 0.4 | 5.5 ± 0.4 | 5.1 ± 0.3 |
| Cl/F (mL/min) | 619.8 ± 36.2 | 641.4 ± 39.1 | 632.4 ± 39.0 | 653.9 ± 37.1 |

*Normalized to a 5 mg dose.

TABLE 4
Pharmacokinetic Parameter Values (Mean and 95% CI) Following Multiple
Oral Doses of Fampridine-SR Tablets (40 mg/day, 20 mg b.i.d.) in 20 Patients
with MS

| | Parameter | | | | |
|---|---|---|---|---|---|
| | Cₘₐₓ | Tₘₐₓ | AUC₍₀₋₁₂₎ | t_{1/2} | Cl/F |
| Day | (ng/mL) | (hours) | (ng · hr/mL) | (hours) | (mL/min) |
| Day 1 | 48.6 (42.0, 55.3) | 3.8 (3.2, 4.3) | NE | NE | NE |
| Day 7/8 | 66.7 (57.5, 76.0) | 3.3 (2.8, 3.9) | 531 (452, 610) | NE | 700 (557, 844) |
| Day 14/15 | 62.6 (55.7, 69.4) | 3.3 (2.6, 3.9) | 499 (446, 552) | 5.8 (5.0, 6.6) | 703 (621, 786) |

NE = Not evaluable

Distribution. The volume of distribution at steady state (V_{dss}) in rats has been reported to approximate total body volume (not adjusted for bioavailability). Following administration of a single p.o. dose of 4-aminopyridine (2 mg/kg) to male and female rats, V_{dss} is 13% lower in females than in males (1094.4 mL in males versus 947.5 mL in females); however, the difference is not statistically significant. Furthermore, when adjusted for body weight differences, there is no difference between males and females (2%).

In a single-dose study, rats were administered ¹⁴C-labeled 4-aminopyridine (1 mg/kg) p.o. Three animals per time point were sacrificed 1, 3, 8, and 24 hours post-dose. Blood was collected and tissues were excised for determination of radioactivity. One hour post-dose, at a time approximately corresponding to the peak plasma concentration, radioactivity was detected in all tissues collected. The amounts represented small percentages of the dose; however, only 58.3% of the dose was accounted for in total. The highest concentrations were in the liver (2.6%), kidney (1.6%), and blood (0.7%); 51% of the radioactivity was in the carcass (primarily the gastrointestinal tract and musculoskeletal system). The half-life of elimination from tissues ranged from 1.1 to 2.0 hours. By 3 hours post-dose, the amount of radioactivity detected in all tissues was negligible (with the exception of the carcass, which contained 15.4% of the radioactive dose).

An in vitro study was conducted to assess plasma protein binding in rat and dog plasma. 4-Aminopyridine concentrations of 5, 50, or 500 ng/mL were used. 4-Aminopyridine was largely unbound and had a high free drug fraction at all three concentrations tested. After a 4-hour dialysis period, the mean percent of free drug ranged from 73 to 94% in rat plasma and 88 to 97% in dog plasma.

Specific studies describing the distribution of 4-aminopyridine across the blood:brain barrier, across the placenta, or into milk have not been identified. However, in the rat, ¹⁴C-labeled 4-aminopyridine was detected in the cerebrum and cerebellum at tissue-to-blood ratios of 3.07 and 1.48, respectively, indicating that 4-aminopyridine crosses the blood brain barrier following an oral dose. 4-aminopyridine is eliminated from the brain at a similar rate as from the blood. Specifically, the elimination half-lives of 4-aminopyridine from brain tissues (cerebellum and cerebrum) and the blood are similar (1.24, 1.63, and 1.21 hours, respectively). Fampridine is largely unbound to plasma proteins (97 to 99%). Administration of a single 20 mg intravenous dose, mean Vd is 2.6 L/kg, greatly exceeding total body water (Uges et al., 1982), similar to values calculated in healthy volunteers and patients with SCI who receive Fampridine-SR tablets. The plasma concentration-time profile is one of two or three compartments with a rapid initial distribution phase. Measurable levels are present in the saliva.

Toxicology. In single- and repeated-dose toxicity studies, the dosing regimen greatly affected the rate of mortality and incidence of clinical signs in all species studied (with the possible exception of the mouse). In general, higher mortality rates and greater incidences of adverse clinical signs were noted when 4-aminopyridine was administered in a single large dose as compared to when the same total dose was given as two, three, or four equally divided sub-doses. Toxic responses to orally administered 4-aminopyridine were rapid in onset, most often occurring within the first 2 hours post-dose.

Clinical signs evident after large single doses or repeated lower doses were similar in all species studied and included tremors, convulsions, ataxia, dyspnea, dilated pupils, prostration, abnormal vocalization, increased respiration, excess salivation, gait abnormalities, and hyper- and hypo-excitability. These clinical signs were not unexpected and represent exaggerated pharmacology of 4-aminopyridine.

In controlled clinical studies involving the use of fampridine, the most frequent adverse events by body system occurred in the nervous system, “body as a whole”, and digestive system. Dizziness, insomnia, paresthesia, pain, headache and asthenia are the most common nervous system adverse events, and nausea is the most frequently reported event in the digestive system category.

The most frequent treatment-related adverse events that have been reported with fampridine-SR, in MS patients as well as other populations including spinal cord injury, may be broadly categorized as excitatory effects in the nervous system, which would be consistent with the potassium channel blocking activity of the compound. These adverse events include dizziness, paresthesias, insomnia, balance disorders, anxiety, confusion and seizure. While an increased incidence of such events appears to be moderately dose-related, the susceptibility of individuals is quite variable. The potential for lowering seizure threshold in people with MS appears to be more significant than for people with spinal cord injury, which may result from interaction of the channel-blocking properties of the drug with MS brain pathology in certain individuals.

Formulations and Administration. It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. Unit dosage forms can be tablets or blister packs. In certain administration protocols a patient may utilize more than a single unit dose at a time, e.g., consume two tablets contained in separate blisters of a blister pack.

Active compounds are administered at a therapeutically effective dosage sufficient to treat a condition associated with a condition in a patient. A “therapeutically effective amount” preferably reduces the amount of symptoms of the condition in the patient by at least about 20%, more preferably by at least about 40%, even more preferably by at least about 60%, and still more preferably by at least about 80% relative to untreated subjects. For example, the efficacy of a compound can be evaluated in an animal model system that may be predictive of efficacy in treating the disease in humans, such as the model systems described herein.

The actual dosage amount of a compound of the present disclosure or composition comprising a compound of the present disclosure administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

Combination Treatments. The compositions and methods of the present invention may be used in the context of a number of therapeutic or prophylactic applications. In order to increase the effectiveness of a treatment with the compositions of the present invention, e.g., aminopyridines, or to augment the protection of another therapy (second therapy), it may be desirable to combine these compositions and methods with other agents and methods effective in the treatment, amelioration, or prevention of diseases and pathologic conditions, for example, cognitive dysfunctions or impairments, ambulatory deficits, etc.

Various combinations may be employed; for example, an aminopyridine or derivative or analog thereof, is “A” and the secondary therapy (e.g., cholinesterase inhibitors such as donepezil, rivastigmine, and galantamine, and immunomodulators such as interferon, etc.) is “B”, nonlimiting combination cycles include:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of a composition of the present invention to a subject will follow general protocols for the administration described herein, and the general protocols for the administration of a particular secondary therapy will also be followed, taking into account the toxicity, if any, of the treatment. It is expected that the treatment cycles would be repeated as necessary. It also is contemplated that various standard therapies may be applied in combination with the described therapies.

Kiss. Kits comprise an exemplary embodiment of the invention. The kit can comprise an outer receptacle or container configured to receive one or more inner receptacles/containers, utensils and/or instructions. A utensil in accordance with the invention can comprise item(s) to administer the drug, such as a patch, inhalation apparatus, fluid container cup, syringe or needle. A composition of the invention can be comprised within a receptacle of the invention. A receptacle of the invention can contain sufficient quantity of a composition of the invention to be useful for multiple doses, or may be in unit or single dose form. Kits of the invention generally comprise instructions for administration in accordance with the present invention. Any mode of administration set forth or supported herein can constitute some portion of the instructions. In one embodiment the instructions indicate that the composition of the invention is to be taken twice daily. The instructions may be affixed to any container/receptacle of the invention. Alternatively, the instructions can be printed on or embossed in or formed as a component of a receptacle of the invention. A kit will also include instructions for employing the kit components as well the use of any other reagent not included in the kit. It is contemplated that such reagents are embodiments of kits of the invention. Such kits, however, are not limited to the particular items identified above and may include any reagent used directly or indirectly in the treatment sought.

### EXAMPLE

Sustained-release fampridine consistently improves walking speed and leg strength in multiple sclerosis and thereby is useful in the treatment of ambulatory deficits associated with MS.

Fampridine (4-aminopyridine) is a potassium channel blocker that has been investigated as a treatment for MS based on a mechanism of increased action potential conduction in demyelinated nerve fibers observed in preclinical studies. Two prior Phase 2 studies (Goodman et al., 2007a, 2008) and a Phase 3 study (Goodman et al., 2007b) of a sustained-release, oral tablet form of fampridine (Fampridine-SR) showed significant improvements in walking and leg strength in MS patients.

A second Phase 3 study of patients with ambulatory deficits due to multiple sclerosis (MS) shows the efficacy and safety of fampridine (Fampridine-SR).

Methods. This was a randomized, double-blind, placebo-controlled, parallel-group study comparing 10 mg sustained-release fampridine (Fampridine-SR) b.i.d. and placebo.

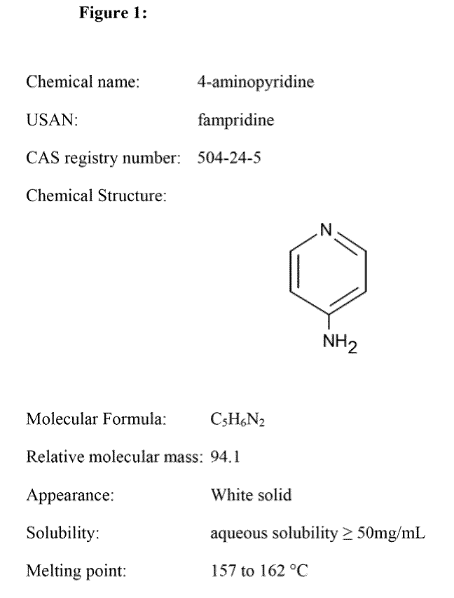

The period of efficacy evaluation (visits 2-6) included 8 weeks of twice daily treatment. An additional week was incorporated to allow pharmacodynamic evaluation at Visit 7, which was not part of the primary endpoint. (See FIG. 1 for study design). In particular, this study is a double-blind, placebo-controlled, parallel group, 13-week study (one week post screening, two weeks of single-blind placebo run-in, eight weeks of double-blind treatment, and two weeks of follow-up) in patients with multiple sclerosis. Approximately 200 patients from approximately 35 centers in the U.S. and Canada are randomized to 10 mg b.i.d. Fampridine-SR or placebo, in a ratio of 1:1 (one patient in the active treatment group to every one patient in the placebo treatment group). One of the goals of this study is to confirm the primary outcome measure in a second prospective study, and equal randomization is the most efficient way to test this outcome. A second follow-up visit four-weeks following final administration of the drug was not included, as it does not appear to provide useful information. A sample size of 92 patients treated with Fampridine-SR 10 mg bid and 92 patients treated with placebo (184 patients in all) provides approximately 90% power, at an overall significance level of 0.05, to detect the difference between a Fampridine-SR 10 mg bid response rate of 30% and a placebo response rate of 10%. In order to ensure at least 184 patients complete the study, approximately 100 patients were randomized to each group.

Key Inclusion Criteria: Clinically definite MS; 18 to 70 years of age; and Able to complete two trials of the Timed 25 Foot Walk (T25FW) within five minutes of each other in 8-45 seconds at screening.

Key Exclusion Criteria: Pregnancy or breast feeding; History of seizure or evidence of epileptiform activity on screening EEG; Previous treatment with fampridine; Onset of MS exacerbation within 60 days prior to screening; Cyclophosphamide, mitoxantrone or (lower extremity) botulinum toxin within 6 months prior to screening; Starting immunomodulatory treatment within 90 days prior, or change in dosing regimen within 30 days prior to screening; or Corticosteroids (other than topical) within 30 days prior, or scheduled corticosteroid treatment during the study; severe renal impairment as defined by a creatinine clearance of <30 mL/minute.

Primary Outcome. The primary outcome was the proportion of patients with consistent improvement in walking speed on the T25FW during the treatment period (those with at least 3 of the 4 on treatment visits with speeds faster than the fastest of the 5 off treatment visits qualified as Timed Walk Responders). The clinical meaningfulness of the response criterion was previously established by correlation with changes in the 12-Item MS Walking Scale (MSWS-12, a patient-reported assessment of walking disability) and both Subject and Clinician Global Impression scales (SGI, CGI).

Secondary Outcome. The prospectively defined secondary outcome was leg strength, measured by Lower Extremity Manual Muscle Test (LEMMT) in 8 muscle groups, comparing the treated Timed Walk Responders and Timed Walk Non-Responders with placebo-treated patients.

Other measures. A number of other measures, not powered for statistical comparison, were included for integrated analyses across studies: MSWS-12, SGI, CGI, Ashworth score.

Summary. The study can be summarized as follows in Table 1.

| | MS-F204 |
|---|---|
| Duration of Treatment | 9 weeks (8 week efficacy period) |
| Exclusion Criteria: Patient has | Included |
| severe renal impairment as | |
| defined by a creatinine | |
| clearance of <30 mL/minute | |
| AE/SAE Reporting Period | 14 days after last dose |
| PK Analysis | Fampridine and metabolite analysis (3-hydroxy 4-aminopyridine and 3-hydroxy |
| | 4-aminopyridine sulfate) |
| Randomization Ratio | 1:1 |
| Statistical Power | 92 FSR: 92 Placebo |
| Primary Measure | To demonstrate that more patients treated with Fampridine-SR 10 mg b.i.d. |
| | experience consistent improvements in walking speed (a validated measure of |
| | clinical meaningfulness) while on drug versus patients treated with placebo. |
| Clinical Meaningfulness | Not required: |
| Secondary Measures | The secondary objectives of the eight-week. double-blind study are to |
| | demonstrate improved leg strength in: |
| | Fampridine-SR 10 mg b.i.d. patients who experience consistent improvements in |
| | walking speed versus placebo; |
| | Fampridine-SR 10 mg b.i.d patients who do not experience consistent |
| | improvements in walking speed versus placebo. Ashworth Score for spasticity |
| | Additional Measures collected but not powered; descriptive statistical analysis |
| | only: |
| | 12-Item Multiple Sclerosis Walking Scale (MSWS-12) |
| | Subject Global Impression (SGI) |
| | Clinician Global Impression (CGI) |
| End of Dosing Interval Visit - | Included |
| to assess declining effect on | |
| efficacy 8-10 hours after last | |
| dose | |

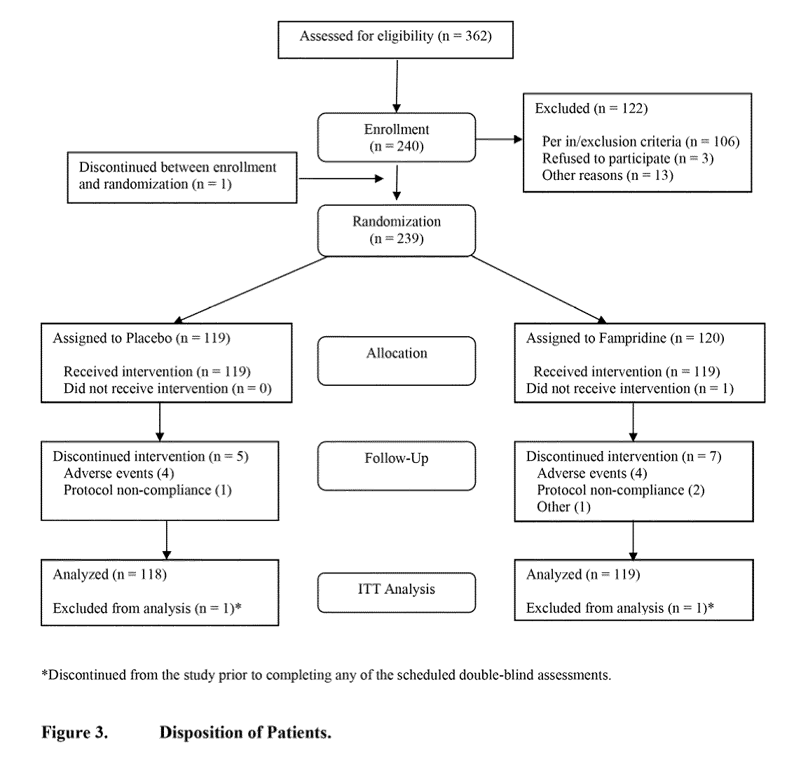

Results. A total of 239 patients were randomized; 120 received fampridine and 119 placebo. 227 patients completed the trial (n=113, 114 for fampridine and placebo respectively). FIG. 3 shows a disposition of patients and Table 2 shows the study demographics.

TABLE 2
Baseline Demographics and Disease Characteristics - Safety Population

| | Placebo | | Fampridine-SR | | |
|---|---|---|---|---|---|
| | (N = 119) | | (N = 120) | | p-value |
| Gender - n (%) | | | | | 0.077 |
| Male | 45 | (37.8%) | 32 | (26.7%) | |
| Female | 74 | (62.2%) | 88 | (73.3%) | |
| Age in years, Mean (SD) | 51.7 | (9.83) | 51.8 | (9.55) | 0.923 |
| Course Type - n (%) | | | | | 0.175 |
| Relapsing-Remitting | 40 | (33.6%) | 43 | (35.8%) | |
| Primary-Progressive | 21 | (17.6%) | 10 | (8.3%) | |
| Secondary-Progressive | 56 | (47.1%) | 62 | (51.7%) | |
| Progressive-Relapsing | 2 | (1.7%) | 5 | (4.2%) | |
| Duration of Disease (years) | | | | | 0.212 |
| Mean (SD) | 13.10 | (8.690) | 14.43 | (9.509) | |
| EDSS Score Mean (SD) | 5.55 | (1.186) | 5.83 | (0.967) | 0.024 |

The slight differences in gender distribution and baseline EDSS score did not affect the efficacy outcomes. P-values for gender and course type from CMH general association test, controlling for pooled center. P-value for age, duration and EDSS from ANOVA models with main effects for treatment group and pooled center.

Efficacy

Timed Walk Response

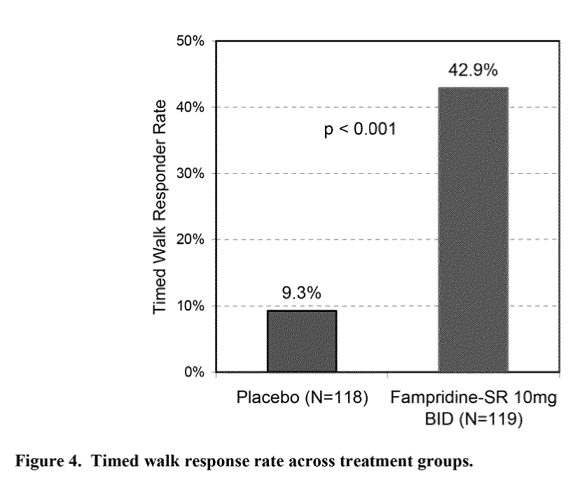

FIG. 4 shows the fampridine-treated group had a higher proportion of Timed Walk Responders, compared to the placebo group. Analyzed by the Cochran-Mantel-Haenszel (CMH) test, controlling for center.

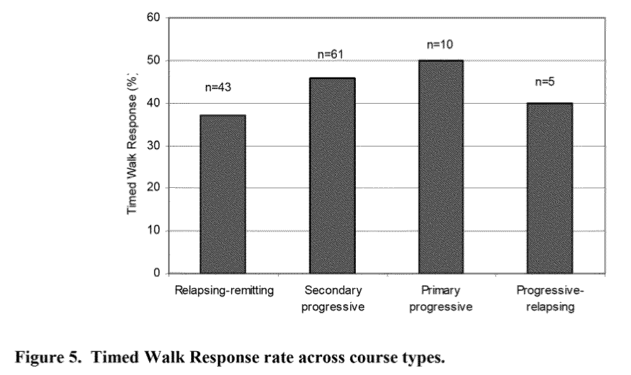

FIG. 5 shows the response rate within the fampridine-treated group was higher across all MS subtypes, and irrespective of whether the patients'were being treated with immunomodulators (42.9% of patients both with (n=70) and without (n=49) concomitant treatment with interferons, natalizumab, or glatiramer acetate).

Change in Walking Speed over Time

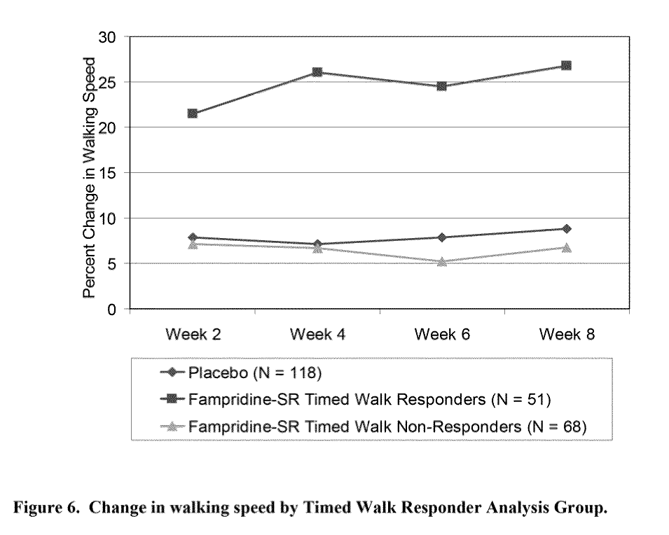

FIG. 6shows walking speed in Timed Walk Responders improved by approximately 25% from baseline, consistently throughout the treatment period.

Change in Leg Strength

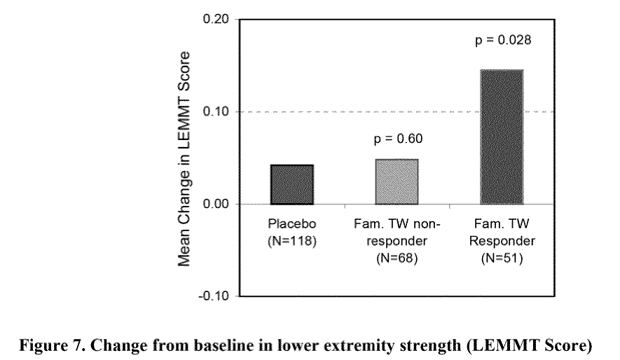

FIG. 7 shows the change in score during the double-blind treatment period is shown by Timed Walk Responder analysis group. (p-values compared to placebo). Leg strength was significantly improved in Timed Walk Responders versus placebo-treated patients (p=0.028). Fampridine-treated Timed Walk Non-Responders were not significantly different from either placebo treated or fampridine Timed Walk Responder groups. (Analyzed by t-tests of the least-squares means using the mean square error via an ANOVA model with effects for responder analysis group and center.)

Other Efficacy Measures

Changes in other measures of efficacy were consistent with previous studies. This included improvement in MSWS-12 score, SGI, and CGI among Timed Walk Responders but not Timed Walk Non-Responders, further validating the clinical meaningfulness of the Timed Walk Response. There was also improvement in the Ashworth Score in the fampridine treated group compared to the placebo treated group, which was significant in an unplanned analysis.

Safety. Documenting Adverse Events: All adverse events reported by the patient or observed by the study personnel during from the Screening Visit to Visit 7 (which is 14 days after the last date of investigational drug administration) were followed and recorded on the Adverse Event Case Report Form, whether or not the event is considered by the Investigator to be related to the investigational drug. Serious adverse events which occur during the study (Screening Visit to Visit 7) or 14 days after the last date of investigational drug administration (if patient discontinued from the study) were captured and reported.

TABLE 3
Treatment-Emergent Adverse Events Occurring in at Least 5% of the
Fampridine-SR Patients (Safety Population)

| | Placebo | | Fampridine-SR | |
|---|---|---|---|---|
| | (N = 119) | | (N = 120) | |
| Patients with at Least One | 79 | (66.4%) | 103 | (85.8%) |
| Treatment-emergent AE | | | | |
| Patients with No Treatment-emergent AE | 40 | (33.6%) | 17 | (14.2%) |

MedDRA Preferred Term

| | | | | |
|---|---|---|---|---|
| Urinary Tract Infection | 10 | (8.4%) | 21 | (17.5%) |
| Fall | 20 | (16.8%) | 14 | (11.7%) |
| Insomnia | 2 | (1.7%) | 12 | (10.0%) |
| Headache | 1 | (0.8%) | 11 | (9.2%) |
| Asthenia | 5 | (4.2%) | 10 | (8.3%) |
| Dizziness | 1 | (0.8%) | 10 | (8.3%) |
| Nausea | 1 | (0.8%) | 10 | (8.3%) |
| Back Pain | 3 | (2.5%) | 7 | (5.8%) |
| Balance Disorder | 2 | (1.7%) | 7 | (5.8%) |
| Upper Respiratory Tract Infection | 8 | (6.7%) | 7 | (5.8%) |
| Arthralgia | 5 | (4.2%) | 6 | (5.0%) |
| Nasopharyngitis | 5 | (4.2%) | 6 | (5.0%) |
| Paraesthesia | 2 | (1.7%) | 6 | (5.0%) |

The majority of adverse events were mild or moderate in intensity and transient. Adverse events were mostly similar to those observed in previous studies of fampridine in MS. The imbalances in the frequency of falls and UTIs were not seen in the previous studies (i.e., MS-F203).

TABLE 4
Serious Treatment-Emergent Adverse Events (Safety Population)

| | Placebo | | Fampridine-SR | |
|---|---|---|---|---|
| | (N = 119) | | (N = 120) | |
| Patients with At Least One Serious | 3 | (2.5%) | 5 | (4.2%) |
| Treatment-emergent AE | | | | |

MedDRA Preferred Term

| | | | | |
|---|---|---|---|---|
| Gastroesophageal Reflux Disease | 1* | (0.8%) | 0 | |
| Chest Discomfort | 1* | (0.8%) | 0 | |
| Cholelithiasis | 0 | | 1 | (0.8%) |
| Cellulitis | 0 | | 1 | (0.8%) |
| Pneumonia | 0 | | 1 | (0.8%) |
| Pyelonephritis | 0 | | 1 | (0.8%) |
| Urinary Tract Infection | 1 | (0.8%) | 0 | |
| Patella Fracture | 0 | | 1* | (0.8%) |
| Syncope | 0 | | 1 | (0.8%) |
| Complex Partial Seizures | 1* | (0.8%) | 0 | |

*Led to discontinuation.

Serious adverse events led to discontinuation in three patients, only one of which (patellar fracture) was in the fampridine group.

Conclusions. Treatment with fampridine was associated with consistently improved walking speed (Timed Walk Response) in a significant proportion (42%) of MS patients during eight weeks of treatment.

This improvement was seen across all MS subtypes and regardless of whether or not the patients were being treated with immunomodulators.

The consistent improvement in walking speed was associated with significant improvements in patient- and clinician-reported outcomes, including the MSWS-12, and both Subject and Clinician Global Impression scales.

Leg strength was improved significantly among Timed Walk Responders.

Safety data were largely consistent with previous experience with fampridine in this population.

Kidney Function. Because fampridine is cleared primarily by the kidneys, proper kidney function is important. Patients with compromised renal function may accumulate excess drug in their bodies. Creatinine clearance is one method of measuring and monitoring kidney function. Accordingly, in some embodiments, the sustained release formulation of 4-aminopyridine is administered to patients having a creatinine clearance rate of at least 30 mL/min. If kidney function is compromised, the dosing level may need to be adjusted or treatment stopped. In some embodiments, kidney function is assessed prior to the first treatment by evaluation of the creatinine clearance rate. To ensure proper kidney function during the course of treatment, additional monitoring may be pursued. In some embodiments, the dose can be reduced to about 5 mg 4-aminopyridine in a sustained release tablet. In other embodiments, the dose can be reduced to about 5 mg or less 4-aminopyridine in a sustained release tablet. As will be appreciated by those of skill in the art, regular monitoring of the creatinine clearance rate will provide an indication of whether kidney function has been compromised. A prescribing physician could then re-evaluate the treatment as needed.

Metabolites. Two primary metabolites of 4-aminopyridine have been found:

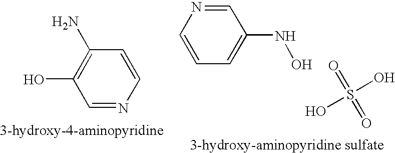

In some embodiments, an effective amount of one or more of the metabolites may be administered to treat ambulatory deficits or other conditions associated with MS. Preferably, such treatments will be administered to renal un-compromised patients having creatinine clearance rates of at least 30 mL/minute. Administration of the metabolite or metabolites may be either direct or via the parent compound. When administered directly, the metabolite or combination of metabolites is administered in a dose equivalent to an effective dose of 4-aminopyridine. In some embodiments, this is a dose equivalent to 10 mg of 4-aminopyridine in a sustained release formulation.

Spasticity. Spasticity is characterized by stiff or rigid muscles with exaggerated, deep tendon reflexes (for example, a knee-jerk reflex). Spasticity generally results from damage to the part of the brain that controls voluntary movement. It may also occur due to damage to the nerves traveling from the brain down to spinal cord, or with the demyelination seen in MS patients. Symptoms of spasticity include: exaggerated deep tendon reflexes (the knee-jerk or other reflexes); scissoring (crossing of the legs as the tips of scissors would close); repetitive jerky motions (clonus), especially when touched or moved; unusual posturing, carrying the shoulder, arm, wrist, and finger at an abnormal angle due to tightness of the muscle. The condition can interfere with walking, movement, or speech. Severe, long-term spasticity may lead to contracture of muscles, causing joints to be bent at a fixed position.

Spasticity may be assessed in addition to walking speed and leg strength. When assessed, spasticity is evaluated at the screening visit and each subsequent visit using the Ashworth Spasticity Score. Preferably, the evaluation is prior to the LEMMT and includes evaluation of six lower extremity muscle groups; knee flexors, knee extensors and hip adductors on both the right and left side of the body. The Ashworth score is obtained prior to LEMMT. For consistency, evaluators should use the same procedures with each visit.

Administration of sustained release 4-aminopyridine may also have beneficial effects in treating spasticity, particularly in the lower extremities. In some embodiments, about 10 mg 4-aminopyridine in a sustained release formulation is administered to an MS patient in need of such treatment. In some embodiments, the patient is renal un-compromised, having a creatinine clearance of at least 30 mL/min. In some embodiments, one or more metabolites of 4-aminopyridine may be administered at dose levels equivalent to the effective dose of the 4-aminopyridine sustained release formulation.

Those of skill in the art will recognize that the methods of treatment disclosed herein may be used in patients suffering from Multiple Sclerosis. More specifically, the methods may be used in treating patients suffering from one of the four main subtypes of MS. In particular, the inventor contemplates a method of treating relapsing-remitting multiple sclerosis in a subject comprising administering a sustained release composition comprising 10 milligrams or less of 4-aminopyridine twice daily to said subject. A further method is contemplated for treating secondary progressive multiple sclerosis in a subject comprising administering a sustained release composition comprising 10 milligrams or less of 4-aminopyridine twice daily to said subject. In particular, the treatment will address ambulatory deficit accompanying MS. Still further is a method of treating primary progressive multiple sclerosis in a subject comprising administering a sustained release composition comprising 10 milligrams or less of 4-aminopyridine twice daily to said subject. In particular, the treatment will address ambulatory deficit accompanying MS. Finally, it is also contemplated that a method of treating progressive-relapsing multiple sclerosis in a subject comprises administering a sustained release composition comprising 10 milligrams or less of 4-aminopyridine twice daily to said subject. In particular, the treatment will address ambulatory deficit accompanying MS.

Although the present invention has been described in considerable detail with reference to certain preferred embodiments thereof, other versions are possible. Therefore the spirit and scope of the appended claims should not be limited to the description and the preferred versions contain within this specification.

What is claimed is:

1. A method of treating multiple sclerosis in a subject comprising administering a sustained release composition comprising 10 milligrams of 4-aminopyridine twice daily to said subject, wherein said multiple sclerosis is selected from relapsing-remitting multiple sclerosis, secondary progressive multiple sclerosis, primary progressive multiple sclerosis and progressive-relapsing multiple sclerosis.

2. The method of claim 1, wherein said sustained release aminopyridine composition comprises one or both of 3-hydroxy-4-aminopyridine and 3-hydroxy-4-aminopyridine sulfate.

3. The method of claim 1, wherein twice daily is every 12 hours.

4. A method of treating multiple sclerosis in a subject comprising
administering to said subject an immunomodulator; and
administering to said subject to said subject a sustained release composition comprising 10 milligrams of 4-aminopyridine twice daily.

5. The method of claim 4, wherein said immunomodulator is selected from interferons, natalizumab, glatiramer acetate an combinations thereof.

6. A method of treating spasticity associated with multiple sclerosis in a subject comprising administering a sustained release composition comprising 10 milligrams of 4-aminopyridine twice daily to said subject, wherein the spasticity of said subject is decreased.

7. A method of treating multiple sclerosis in a subject comprising
measuring said patient's creatinine clearance; and
administering a sustained release composition comprising 10 milligrams of 4-aminopyridine twice daily to said subject if said subject's creatinine clearance is greater than or equal to 30 ml/min.

8. The method of claim 7, wherein measuring said patient's creatinine clearance occurs prior to initial administration of said sustained release composition comprising 10 milligrams of 4-aminopyridine.

9. The method of claim 7, wherein measuring said patient's creatinine clearance occurs during a treatment period.

10. The method of claim 7, wherein administering a sustained release composition comprising 10 milligrams of 4-aminopyridine twice daily to said subject is continued unless said subject's creatinine clearance is less than 30 mL/min.

11. A method of treating multiple sclerosis multiple sclerosis in a subject comprising
measuring said patient's creatinine clearance; and
administering a sustained release composition comprising 4-aminopyridine, wherein the amount and the frequency of administration to said patient is dependent upon the measured creatinine clearance.

12. A method of testing the efficacy of a sustained release composition comprising 4-aminopyridine for treating multiple sclerosis comprising:
assessing potential patients for study, based on particular inclusion and exclusion criteria, excluding patients with creatinine clearance rates below about 30 mL/min;
assigning known portions of patients to placebo and Fampridine-SR groups, unknown to them or an evaluator in a double-blind study for receipt of placebo or Fampridine-SR; and
assessing one or more of walking speed, leg strength, and spasticity over the course of 8 weeks of treatment.

13. The method of claim 12, wherein creatinine clearance rates are obtained prior to each assessment.

14. A method assessing the efficacy of a sustained release composition comprising 4-aminopyridine for treating multiple sclerosis comprising
assigning known portions of a sample of patients with multiple sclerosis to placebo and Fampridine-SR groups, unknown to them or an evaluator in a double-blind study for receipt of placebo or Fampridine-SR; and
assessing one or more of walking speed, leg strength, and spasticity for said patients over the course of treatment;
wherein the size of said sample of patients shall provide about 90% power and a statistical significance level of 0.05 or lower.

15. The method of claim 14 further comprising assessing potential patients for study based on particular inclusion and exclusion criteria.

16. The method of claim 15, wherein said exclusion criteria is a creatinine clearance rate below about 30 mL/min.

17. The method of claim 14, wherein the course of treatment is eight weeks.
